(19) United States
(12) Patent Application Publication
(10) Pub. No.: US 2006/0212114 A1
(43) Pub. Date: Sep. 21, 2006

(54) Devices and methods for mitral valve annulus reformation

(75) Inventors: Lorenzo Menicanti, Sergrate Milan (IT); Mitta Suresh, Richardson, TX (US); Jill Giannoble, Plano, TX (US); Alan Bachman, Milford, CT (US); Ernie Corrao, Bethel, CT (US)

Correspondence Address: MEYERTONS, HOOD, KIVLIN, KOWERT & GOETZEL, P.C., 700 LAVACA, SUITE 800, AUSTIN, TX, 78701, US

(73) Assignee: Chase Medical, L.P., Richasrdson, TX (US)

(21) Appl. No.: 10/530,938

(22) PCT Filed: Oct. 8, 2003
(86) PCT No.: PCT/US03/31992

§ 371 Date: Apr. 8, 2006

Related U.S. Application Data
(63) Continuation of application No. 09/360,709, filed on Jul. 26, 1999, now Pat. No. 6,610,071.
(60) Provisional application No. 60/421,712, filed on Oct. 28, 2002.
(60) Provisional application No. 60/416,482, filed on Oct. 8, 2002.

(51) Int. Cl. A61F 2/24

(52) U.S. Cl. 623/2.36; 600/201

(57) ABSTRACT

A method and apparatus to allow a user to quickly and inexpensively repair the dilation of the mitral annulus is presented. In some embodiments, the method and apparatus should allow the user to reduce the size of the annulus to a predetermined size and allow him to secure the annulus so that it remains at the selected size. An apparatus may include a retractor. The retractor may assist in positioning a portion of the annulus. The apparatus may be part of a system including one or more fasteners. The fasteners may inhibit deformation of the reduced diameter of the mitral valve annulus. In some embodiments, the method may include incising a portion proximate to the mitral valve annulus. The method may include positioning a device through the incision, attaching the device with a plurality of attachment members, and deforming the device while observing the annulus and a surrounding area.

### BACKGROUND

1. Field of the Invention

This invention relates generally to devices and methods for mitral valve annulus reformation.

2. Description of the Related Art

The function of a heart in an animal is primarily to deliver life-supporting oxygenated blood to tissue throughout the body. This function is accomplished in four stages, each relating to a particular chamber of the heart. Initially deoxygenated blood is received in the right auricle of the heart. This deoxygenated blood is initially received in the left auricle of the heart and ultimately pumped by the left ventricle of the heart throughout the body. It may be seen that the left ventricular chamber of the heart is of particular importance in this process as it is relied upon to pump the oxygenated blood initially through a mitral valve into the left ventricle and out through the aortic valve and ultimately throughout the entire vascular system.

The shape and volume of the normal heart are of particular interest as they combine to dramatically affect the way that the blood is pumped. The left ventricle which is the primary pumping chamber, is somewhat elliptical, conical or apical in shape in that it is longer than it is wide. The longest portion of the long axis of the left ventricle is generally from the aortic valve to the apex of the left ventricle. The widest portion of the short axis of the left ventricle is generally from the ventricle wall to the septum. The left ventricle descends from a base with a decreasing cross-sectional circumference, to a point or apex. The left ventricle is further defined by a lateral ventricle wall and a septum, which extends between the auricles and the ventricles. The left ventricle also contains, and its performance is affected by, the aortic valve and parts of the mitral valve apparatus, papillary muscles, chordae tendinaea, mitral annulus and valve leaflets.

The contribution of the aortic and mitral valves to the left ventricle performance makes them crucial to the overall performance of the ventricle. These valves control how much blood can come into or out of the ventricle. In particular the mitral valve is critical in its effect on the ventricle, since the mitral valve apparatus aids in the motion of the heart, by helping move the apex closer to the annulus. By closing properly, the mitral valve leaflets ensure that all the oxygenated blood flow is ejected through the aortic valve. If the leaflets are disfigured in some way they may let oxygenated blood flow backward through the valve into the left atrium, depriving the body of needed oxygenated blood. Similarly if the aortic valve is damaged or does not function properly, blood pressure falls as blood leaks from the aorta into the ventricle, or the valve cannot open far enough to let sufficient blood flow into the body.

Many patients who have mitral valve regurgitation, retrograde flow of blood back through the mitral valve, only have dilation of the mitral valve annulus. Most patients with congestive heart failure do not have congenital valve disease. The majority of patients with congestive heart failure and mitral valve regurgitation have dilated mitral valve annuluses. The dilation of the ventricle in congestive heart failure causes the annulus, which is attached to the free wall of the ventricle, to expand outwards along with the free wall of the ventricle. Mitral valve annulus dilation is generally in the posterior region. FIG. 1 depicts an embodiment of a dilated mitral valve annulus thru a left ventricle. The anterior region between trigone and trigone generally does not dilate.

Various surgical approaches have been taken to repair the dilated annulus of the mitral valve. Surgical procedures have been developed to shorten the annulus to reduce the dilation. Generally these procedures involve placing a reinforced flexible ring (annuloplasty ring) around the annulus and sometimes, in addition, shortening the size of the mitral valve posterior leaflet.

The placement of an annuloplasty ring is most easily done when approaching the mitral valve through the left atrium or right atrium across the septum. This approach gives the user direct access to the mitral valve annulus. The current standard repair of the annulus is time consuming and costly. The current standard reinforcement device (annuloplasty ring) sells for approximately $1,000-$2,000 and requires around 10 to 15 sutures to put securely in place. The expense of the sutures adds to the cost of the procedure, as does the amount of time needed to place and secure the multitude of sutures which adds to the total amount of time the patient needs to be on cardiopulmonary bypass. Increasing the length of the bypass run has been proven to lead to complications and detrimental effects to the patient.

When surgical ventricular restoration is performed, the left ventricle is opened. The access to the valve is usually more constrained from this opening. The usual ventricle-opening site places the mitral annulus down and to the right of the surgical opening. It also leaves some overhanging parts of the ventricle over the mitral valve to partially obscure the annulus. The papillary muscles and chordae tendinae are also in the left ventricle. These take up room and help to obscure the view of the valve. They also would interfere with the placement of a bulky device, such as an annuloplasty ring. Gaining access to the valve during a surgical ventricular repair procedure would require repositioning the heart, and making another incision into the heart right atrium, thereby lengthening and complicating the overall procedure and inducing more trauma to the patient

Currently the amount of repair that has to be done to the annulus is determined once the annulus is accessed. Then users estimate how much to reduce the annulus size. Preoperatively an image may be made of the ventricle and converted into a three-dimensional image that may be manipulated through the use of an elastance model. This elastance model will replicate how the mitral annulus will shorten when placed under pressure of the sutures. The model may then tell the user how large the valve opening is and how much suture had to be pulled to get to that opening size. The user may have the model create the size opening he desires and then transfer the length of suture pulled in the simulation to the suture length actually used in surgery.

What is needed therefore is a reliable method and apparatus to allow a user to quickly and inexpensively repair the dilation of the mitral annulus. The apparatus should allow the user to reduce the size of the annulus to a predetermined size and allow him to secure the annulus so that it remains at the selected size. In response to these and other problems, an improved apparatus and method is provided for repairing the dilation of a mitral valve annulus.

### SUMMARY

In an embodiment, a mitral valve annulus reformation may be achieved with a single suture, pledgets, and a sizer. These devices will provide the same effect as the current procedure while greatly reducing the time and cost needed to make the repair. These devices may be used for a stand-alone mitral valve repair or in combination with another procedure such as surgical ventricular repair.

In some embodiments, a suture with color-coded bands, a pledget (made of felt, for example) secured to a portion of the suture (for example, the middle of the suture), another pledget secured to another portion of the suture, and a sizer are provided to allow a user to quickly and inexpensively repair the dilation of the mitral valve annulus.

In certain embodiments, a method for reducing a size of a mitral valve annulus comprises positioning a plurality of first shaped staples along a portion of a circumference of the annulus. This causes the first shaped staples to form second shaped staples upon penetration of the circumference, thereby reducing the portion of the circumference of the annulus.

In some embodiments, a method for reducing the size of the mitral valve annulus includes positioning at least one object adapted to engage a portion of a circumference of the annulus, engaging the portion, and deforming the object. The deformation may cause a reduction of the portion.

In certain embodiments, a method for repairing a mitral valve annulus includes placing a tool in the mitral valve. The annulus around the tool may be tightened with a suture.

In some embodiments, similar results to current procedures may be achieved by utilizing a single adjustable clip. Such a clip will provide a similar effect as the current procedure while greatly reducing the time and cost needed to make the repair. A clip may be placed externally in the atrioventricular (AV) groove (or coronary groove) of the heart by incising the fatty tissue or fat pad of the AV groove along the posterior annulus of the mitral valve. A clip may be used for a stand-alone mitral valve repair or in combination with another procedure such as surgical ventricular repair.

In one embodiment, a method for reducing a size of a mitral valve annulus may include incising a portion of a fatty tissue in an atrioventricular groove proximate to the mitral valve annulus. A device may be positioned through the incision and proximate to a portion of a great cardiac vein. The device may include a plurality of attachment members. The attachment members may be attached to a portion of the mitral valve annulus. Upon attachment of the attachment members to a portion of the mitral valve annulus, the device may be deformed. In some embodiments, the device may be deformed under echocardiography. If negative results are indicated from the echocardiography, the device is further deformed.

In another embodiment, a method for reducing a size of a mitral valve annulus may include incising a portion of the great cardiac vein. A device may be positioned through the incision. The device may be deformed. The deformation may reduce the size of the annulus.

In some embodiments, a device adapted to reduce a size of a mitral valve annulus may include an approximately semicircular adjustable body. The device may include a plurality of attachment members coupled to one side of the body. The device may be adapted to be deformed. Deformation of the device may cause an engagement of the annulus by the attachment members reducing the size of the annulus.

In certain embodiments, a device adapted to reduce a size of a mitral valve annulus includes a first portion, a second portion, and a third portion. The first portion and the second portion may be slidably attached to the third portion. The third portion may be adapted to secure the first portion and the second portion. The first portion and the second portion may include a plurality of attachment members on similar sides.

In some embodiments, a method for incising an atrioventricular (AV) groove may include incising the AV groove via a scalpel. The scalpel may be adapted to blindly incise the AV groove. The method may include viewing the incision via a reflective surface adapted to provide a viewable incision area of the AV groove.

### BRIEF DESCRIPTION OF THE FIGURES

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts an embodiment of a dilated mitral valve annulus thru a left ventricle.

FIG. 2 depicts an embodiment of a tightening suture proximate to the annulus.

FIG. 3 depicts an embodiment of a tightened suture and annulus.

FIG. 4 depicts an embodiment of a sizer.

FIG. 5 depicts an embodiment of a suture.

FIG. 6A depicts an embodiment of an enlarged mitral valve annulus and first staple.

FIG. 6B depicts an embodiment of a reformed mitral valve annulus and second staple.

FIG. 7A depicts an embodiment of an annular stent before activation.

FIG. 7B depicts an embodiment of an annular stent after activation.

FIGS. 8A-C depict a perspective view of an embodiment of a repair instrument, a sizer, and a repaired mitral valve annulus respectively.

FIGS. 9A-B depict a perspective view of an embodiment of a retractor during use.

FIGS. 10A-B depict a perspective view of an embodiment of a retractor during use.

FIG. 11 depicts a crosscut view of an embodiment of a retractor during use.

FIGS. 12A-B depict a perspective view of an embodiment of a retractor during use.

FIGS. 13A-D depict a perspective view of an embodiment of a retractable suture needle fastener system.

FIG. 14 depicts a crosscut view of an embodiment of a retractable suture needle fastener system.

FIG. 15 depicts a perspective view of an embodiment of a retractable suture needle fastener system.

FIGS. 16A-B depict a perspective view of an embodiment of a clamping fastener system.

FIGS. 17A-B depict a perspective view of an embodiment of a clamping fastener system.

FIGS. 18A-B depict a perspective view of the distal end of an embodiment of a collet fastener system.

FIGS. 19A-B depict a cross sectional view of the distal end of an embodiment of a collet fastener system.

FIG. 20 depicts a perspective view of a mitral valve annulus after removal of an embodiment of a collet fastener system.

FIGS. 21A-B depict a cross sectional view of the distal end of an embodiment of a collet fastener system.

FIGS. 22A-B depict a perspective view of an embodiment of a fastener system.

FIG. 23 depicts a perspective view of a mitral valve annulus after removal of an embodiment of a collet fastener system.

FIGS. 24A-B depict a cross sectional view of the distal end of an embodiment of a collet fastener system.

FIG. 25 depicts a perspective view of a mitral valve annulus after removal of an embodiment of a retractor system.

FIGS. 26A-B depict a cross sectional view of the distal end of an embodiment of a collet fastener system.

FIG. 27 depicts a perspective view of a mitral valve annulus after removal of an embodiment of a retractor system.

FIGS. 28A-B depict a cross sectional view of the distal end of an embodiment of a collet fastener system.

FIG. 29 depicts a perspective view of a mitral valve annulus after removal of an embodiment of a retractor system.

FIGS. 30A-B depict a cross sectional view of the distal end of an embodiment of a collet fastener system through a mitral valve annulus.

FIG. 31 depicts a perspective view of a mitral valve annulus after removal of an embodiment of a retractor system.

FIGS. 32A-B depict a cross sectional view of the distal end of an embodiment of a collet fastener system.

FIG. 33 depicts a perspective view of a mitral valve annulus after removal of an embodiment of a retractor system.

FIGS. 34A-E depict a cross sectional view of the distal end of an embodiment of a collar fastener system.

FIGS. 35A-E depict a cross sectional view of the distal end of an embodiment of a staple fastener system.

FIGS. 36A-C depict a cross sectional view of the distal end of an embodiment of a staple fastener system.

FIG. 37 depicts a perspective view of a mitral valve annulus after positioning a system of annulus reducing fasteners.

FIG. 38 depicts a view of a coronary sinus in relation to a mitral valve annulus.

FIG. 39 depicts a view of an incision along the coronary sinus.

FIG. 40A depicts a view of an embodiment of a first clip.

FIG. 40B depicts a view of the first clip after deformation.

FIG. 41A depicts a view of a second clip in an initial position.

FIG. 41B depicts a view of the second clip in an adjusted position.

FIG. 42 depicts a view of a scalpel.

FIG. 43 depicts a view of a mirror.

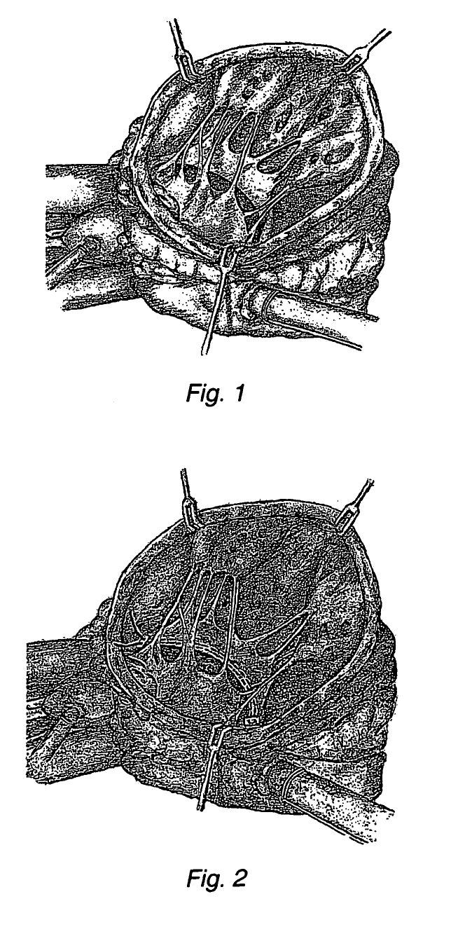

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION

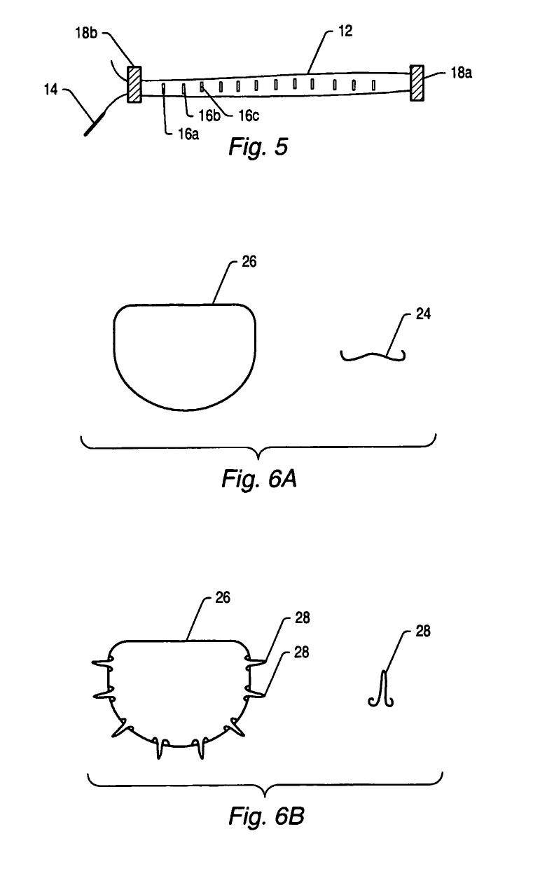

In some embodiments, a method may address the ability of a user to quickly and accurately repair a dilation of a mitral valve annulus. Reparation of a dilated mitral valve annulus may be accomplished by using a sizing tool or sizer 10 (depicted in FIG. 4). Sizer 10 may be placed in mitral valve opening 20 to ensure that the repair creates the correctly sized opening. The annulus may be tightened around the sizer with a suture 12 (depicted in FIG. 5). Suture 12 may include at least one of the following items: suture needle 14, one or more markers 16 along its length or along a portion of its length, and one or more pledgets 18. Suture needles 14 may be located at each end. Pledget 18 may be substantially fixed to suture 12. Pledget 18 may be substantially fixed at a midpoint of suture 12. Suture 12 may include an additional pledget secured at the end opposite from first fixed pledget 18a.

Systems and devices described herein may be made of various materials including, but not limited to, metals, metal alloys, silicon, plastic, polymers, ceramics, and combinations thereof. Some systems and devices may include components made of materials that can be autoclaved and/or chemically sterilized. Some components of a systems and devices may be formed of materials unable to be autoclaved and/or chemically sterilized. Components unable to be autoclaved and/or chemically sterilized may be made of sterile materials and placed in working relation to other sterile components during assembly of a systems and devices. In some embodiments, an entire assembly may be made of materials that can be autoclaved and/or chemically sterilized so that the assembly is a reusable instrument. In other assembly embodiments, all or selected components of the assembly may be made of sterile, disposable materials so that the selected components of the assembly are designed for single use.

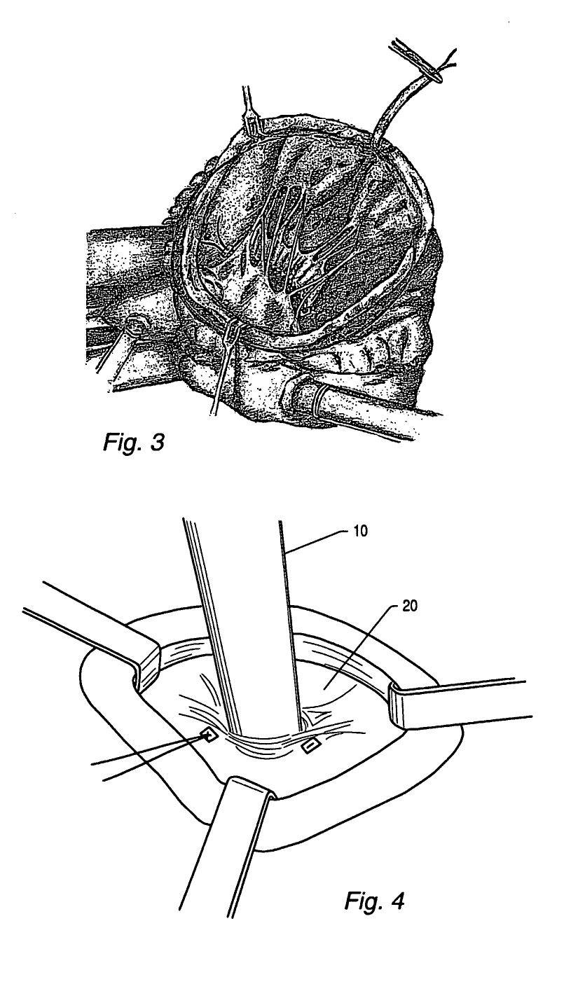

In some embodiments, a method for reparation of a dilated mitral valve annulus may involve the user gaining access to the mitral valve either through the left or right atriums and/or by opening the left ventricle in an akinetic area. The user may weave one end of suture 12 from trigone to trigone on the posterior side of the mitral annulus. The opposite end of suture 12 may be woven from trigone to trigone on the posterior side of the mitral annulus. Both suture lines may be placed adjacent to one another on the posterior side of the annulus. Fixed pledget 18a may remain adjacent to the trigone where the user initiated the suturing. Remaining pledget 18b may be put over suture needles 14 and run down suture 12. The user may insert correct sizer 10 into mitral valve opening 20, as shown in FIG. 4. The user may push pledget 18b down the sutures, shortening and plicating the mitral annulus until it fits snugly against the sizer. The user may note which marker 16 on which pledget 18b rests and remove sizer 10. By noting on which marker 16 pledget 18b sits, the user may ensure that he does not make the opening any smaller or larger when suture 12 is secured without sizer 10 in place. If pledget 18b moves from the original color band, the user may tell if the opening has been made too small or if the opening has expanded to a larger than desired size. With sizer 10 removed, the user has room to secure pledget 18b and suture 12. Markers 16 also ensure that the user is pulling equally on the ends of suture 12. By ensuring that pledget 18b rests on the same color band on each strand of suture 12, the user knows that the annulus has not been tightened unevenly. Tightening the annulus unevenly may upset the natural saddle shape of the annulus and it may affect the natural orientation of the annulus. FIG. 2 depicts an embodiment of a tightening suture proximate to the annulus. FIG. 3 depicts an embodiment of a tightened suture and annulus.

In some embodiments, suture 12 may be made of standard suture material and suture needles. Markers 16 may be alternating colors of at least one different color, but preferably two or more different colors. Markers 16 may also be a set distance between each other. For example, markers 16 may be about 0.5 cm apart from each other. In some embodiments, markers 16 may be set any reasonable distance apart from each other that may be useful for a user. This allows the user to calculate how much valve opening 20 is contracted. The overall length of suture 12 may be around 50.00 cm. The sizes of sutures 12 may be standard suture sizes. In some embodiments sutures 12, may come in patient specific sizes. Pledgets 18 may be formed from felt or a similar material. One pledget 18 may be secured to the midsection of suture 12.

Sizer 10 may be of many different configurations. In some embodiments, sizer 10 may be adjustable from the smallest size to the largest size. In some embodiments, sizer 10 may be a set of sizers sold together with various sizes. Sizer 10 may be made out of any rigid and/or semirigid material such as plastic, stainless steel, etc. In certain embodiments, sizer 10 may be about 22 mm, 24 mm, and/or 26 mm in diameter. In some embodiments, sizer 10 may be between about 15 mm and about 32 mm in diameter.

In some embodiments, pledgets 18 maybe made of any soft biocompatible material (e.g., felt). In certain embodiments, pledgets 18 may have one or more different shapes (e.g., rectangular, square, circular, or figure eight). Pledgets 18 may have one or more predrilled holes to ease placing suture needle 14 through.

FIG. 5 depicts an embodiment of suture 12 having three different colored markers 16. A distance of about 0.5 cm separates markers 16. In some embodiments, uniform, non-uniform, smaller, or larger number of colors and distances between markers 16 may be used with the suture. In some embodiments, suture 12 comprises needles 14 on each end of the suture, colored measurement markers 16 along a length of the suture, pledget 18a fixed at a midpoint of the suture, and pledget 18b adapted to be secured at an opposite end of the fixed pledget 18a.

In some embodiments, suture 12 may include attachment members. Attachment members may be on at least one end of the suture. Markers 16 may be proximate to the at least one end of suture 12. First pledget 18a may be proximate to markers 16. Second pledget 18b may be positioned at an opposite end of first pledget 18a. In some embodiments, suture 12 may include attachment members, markers 16, and at least one pledget 18.

FIG. 6A depicts an enlarged mitral valve annulus and first shaped staple 24. Staple 24 may be formed of various uniform and non-uniform shapes, sizes, and/or materials. In some embodiments, a method for reducing a size of mitral valve annulus 26 includes positioning a tool (not shown) along a portion of a circumference of the annulus. The tool may include one or more first shaped staples 24. The tool may be activated upon properly positioning the tool adjacent the portion of the circumference of annulus 26. Activating the tool causes first shaped staples 24 to form second shaped staples 28 (see FIG. 6B) upon penetration of the circumference. Penetration of the circumference and formation of second shaped staples 28 reduces the portion of the circumference of annulus 26.

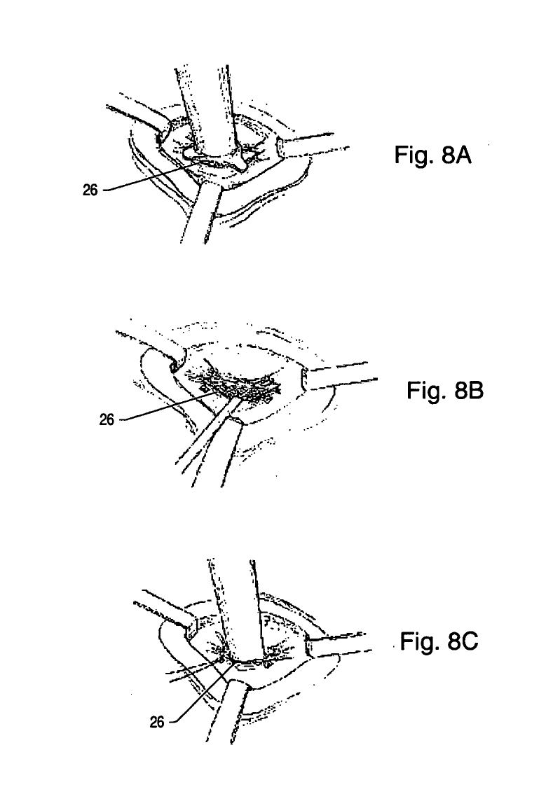

In some embodiments, a method for reducing a size of mitral valve annulus 26 may include positioning a plurality of first shaped staples 24 along a portion of a circumference of annulus 26. The method may include activating first shaped staples 24 to form second shaped staples 28 upon penetration of the circumference of annulus 26, thereby reducing the portion of the circumference of annulus 26. In certain embodiments, a method for reducing a size of mitral valve annulus 26 may include positioning at least one object along a portion of annulus 26, and causing the at least one object to reduce the portion of annulus 26 as depicted, for example, in FIGS. 8A-C. In some embodiments, a method for reducing a size of mitral valve annulus 26 may include reducing a size of an annulus 26 by an object.

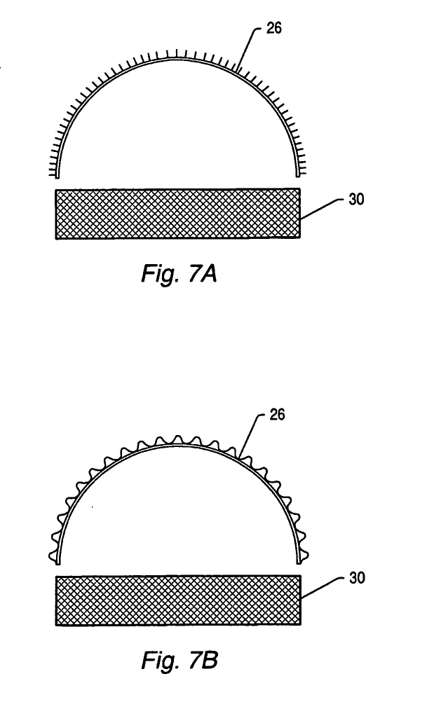

FIGS. 7A and 7B depicts annular stents 30 before and after activation. Stent 30 may be matched to an enlarged annulus 26. Stent 30 may be formed of various uniform and non-uniform shapes, sizes, and/or materials. Stent 30 may be expandable and/or retractable. In some embodiments, stent 30 may be a thin strip of material (e.g., a mesh-like material). In some embodiments, a method for reducing the size of mitral valve annulus 26 may include positioning stent 30 proximate to a portion of a circumference of annulus 26. Stent 30 may include attachment members (not shown). The attachment members may facilitate positioning stent 30 proximate to a portion of a circumference of annulus 26. The method may include positioning the attachment members through tissue along the portion of a circumference of annulus 26 in order to substantially couple stent 30 to the portion. Upon coupling stent 30 to the portion of a circumference of annulus 26 using the attachment members, the stent may be deformed. Deforming stent 30 will cause a reduction of the portion of a circumference of annulus 26.

In some embodiments, a method for reducing the size of mitral valve annulus 26 may include locating and/or exposing a portion of the circumference of annulus 26. The method may include coupling fasteners to at least a portion of the circumference of annulus 26. In some embodiments, coupling fasteners to a portion of a circumference of annulus 26 may assist in reducing the size of mitral valve annulus 26. In some embodiments, fasteners coupled to a circumference of annulus 26 may reduce the size of mitral valve annulus 26 upon activation. In certain embodiments, fasteners coupled to a circumference of annulus 26 may act as a coupling means for a device capable of reducing the size of mitral valve annulus 26.

In certain embodiments, a method fbr reducing the size of mitral valve annulus 26 may include locating and/or exposing a portion of the circumference of annulus 26. The portion of the circumference of annulus 26 may include the fibrous ring forming the circumference. Locating and/or exposing the portion of the circumference may include retracting the posterior leaflet of the mitral valve. Retracting the posterior leaflet of the mitral valve may include positioning portions of a retractor 32 adjacent to a posterior leaflet 34. Once positioned adjacent posterior leaflet 34, retractor 32 may be activated extending and/or retracting the posterior leaflet. In some embodiments, fasteners 36 may be coupled to a portion of a circumference of annulus 26 upon retraction of posterior leaflet 34 in order to facilitate reduction of the size of mitral valve annulus 26.

In some embodiments, a method for reducing the size of mitral valve annulus 26 may include positioning at least one object adapted to engage a portion of a circumference of annulus 26, engaging the portion, and deforming the at least one object, wherein the deformation causes a reduction of the portion. In certain embodiments, a method for reducing the size of mitral valve annulus 26 may include positioning an object proximate to the annulus, and activating the object, wherein the activation causes a reduction of the annulus.

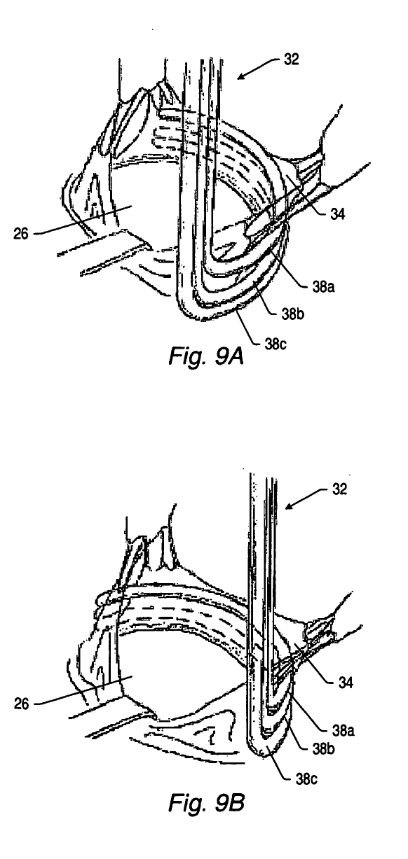

FIGS. 9-11 depict a distal portion of an embodiment of a retractor 32 during use. Retractor 32 may include a plurality of support members 38. Some embodiments of retractor 32 may include three support members 38a-c as depicted in FIGS. 9-11. Support members 38a-c may be composed of any appropriate rigid and/or semi-rigid material known to one skilled in the art (e.g., stainless steel or polymers). The distal ends of support members 38 may be curved into a semicircular shape in a plane perpendicular to the proximal portion of support members 38. Each support member 38 may be formed from one single continuous piece of material. In other embodiments, one or more support members 38 may be formed from multiple pieces coupled together. Support members 38 may be coupled to one another. Support members 38 may be coupled to one another to maintain a specific alignment and/or orientation to one another. Support members 38 may be coupled to one another while still allowing axial movement relative to one another. Means may be provided to limit axial movement of support members 38 relative to one another. Means for limiting axial movement may assist in preventing over extension and damage to posterior leaflet 34.

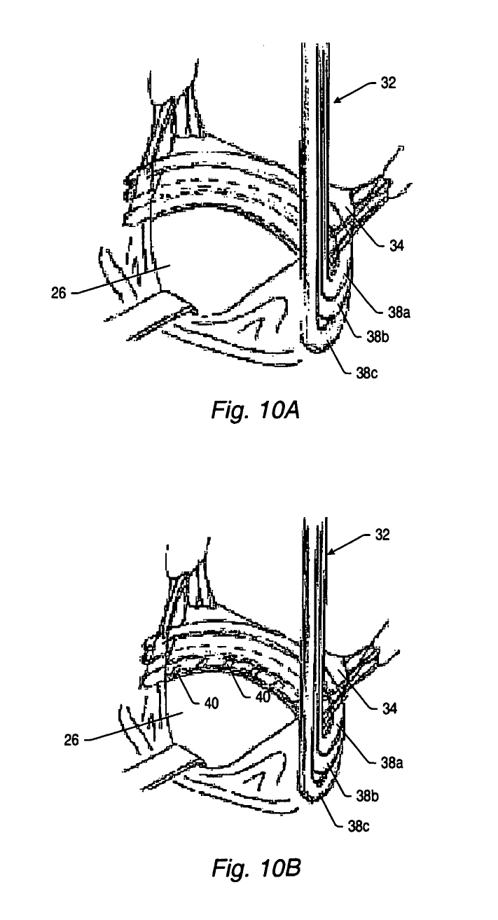

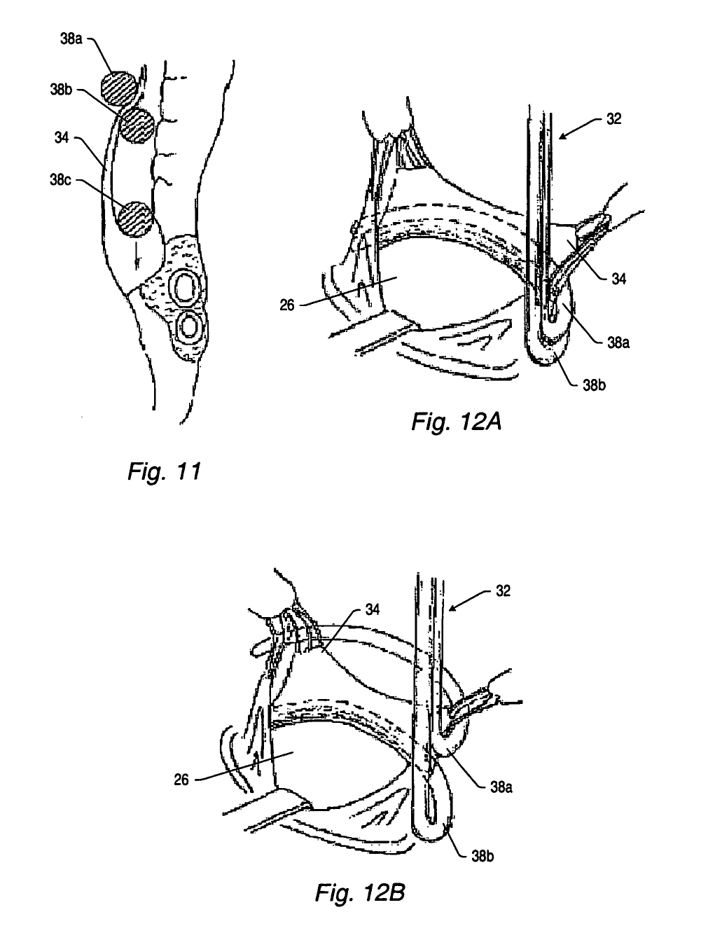

During a procedure, the distal end of support members 38a-c may be positioned (e.g., rotated) into place adjacent posterior leaflet 34. Support members 38b-c may be positioned behind and/or below posterior leaflet 34. Support member 38a may be positioned in front and/or above posterior leaflet 34 as depicted in FIG. 9A. Support members 38a-b are positioned to apply pressure on posterior leaflet 34 to inhibit any undesirable movement while allowing for controlled movement and positioning of the posterior leaflet as depicted in FIG. 9B. Support members 38a-b may be moved axially in a direction opposite support member 38c as depicted in FIG. 11. Moving support members 38a-b in such a manner may stretch portions of posterior leaflet 34 as depicted in FIG. 10A. Stretching portions of posterior leaflet 34 may delineate portions of the fibrous ring forming mitral valve annulus 26 allowing a user too more easily identify the fibrous ring.

In some embodiments, support member 38c may include a plurality of openings (e.g., suction ports 40 as depicted in FIG. 10B). Suction ports 40 may be coupled through support member 38c to a vacuum producing mechanism capable of drawing a controlled vacuum through suction ports 40. Drawing a vacuum through suction ports 40 may further delineate portions of the fibrous ring forming mitral valve annulus 26. Drawing a vacuum through suction ports 40 may facilitate the use of suction ports 40 as suture guides. Drawing a vacuum through suction ports 40 may assist in inhibiting movement of lower portions of posterior leaflet 34.

FIGS. 12A-B depict a perspective view of an embodiment of retractor 32 during use. Retractor 32 may include two support members 38a-b as depicted in FIG. 12A-B. During a procedure, the distal end of support members 38a-b may be positioned (e.g., rotated) into place adjacent posterior leaflet 34. Support member 38b may be positioned behind and/or below posterior leaflet 34. Support member 38a may be positioned in front and/or above posterior leaflet 34, as depicted in FIG. 12A. Once support members 38a-b are positioned, support member 38a may be moved axially in a direction opposite support member 38b, as depicted in FIG. 12B. Moving support member 38a in such a manner may stretch portions of posterior leaflet 34, as depicted in FIG. 12B. Stretching portions of posterior leaflet 34 may delineate portions of the fibrous ring forming mitral valve annulus 26 allowing a user too more easily identify the fibrous ring.

In some embodiments, movement of support members of a retractor may be controlled by a mechanism. The mechanism may include means known to one skilled in the art for positioning the support members relative to one another in small increments in a controlled fashion (e.g., a ratchet system).

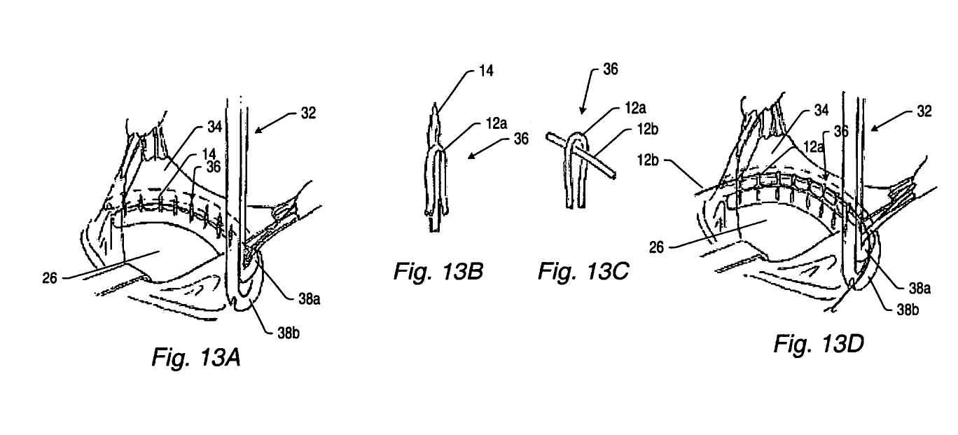

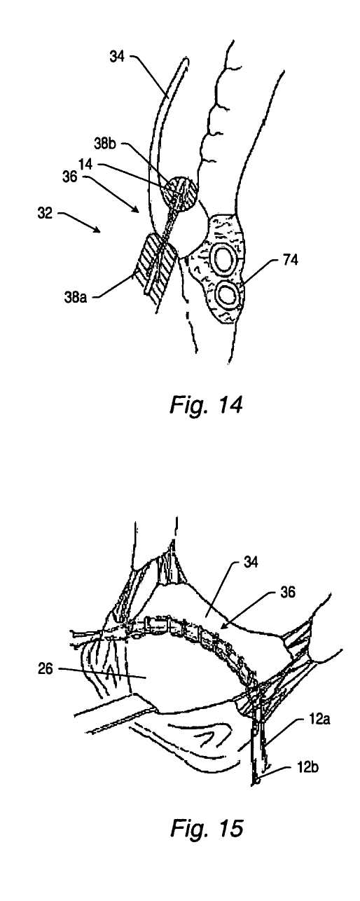

In some embodiments, a system of fasteners 36 may be employed to assist in reducing the size of mitral valve annulus 26. In certain embodiments, a method for reducing the size of mitral valve annulus 26 may combine the use of retractor 32 and a system of fasteners 36. FIGS. 13-15 depict an embodiment of a device combining the concept of retractor 32 and a system of fasteners 36. Retractor 32 may include two support members 38a-b as depicted in FIGS. 13A-B. During a procedure, the distal end of support members 38a-b may be positioned (e.g., rotated) into place adjacent posterior leaflet 34. Support member 38b may be positioned behind and/or below posterior leaflet 34. Support member 38a may be positioned substantially within annulus 26 and in front of posterior leaflet 34 as depicted in FIG. 13A. Once support members 38a-b are positioned, support member 38a may be moved axially in a direction toward support member 38b as depicted in FIG. 13A. Moving support member 38a in such a manner may inhibit movement of portions of a circumference of annulus 26 (e.g., the fibrous ring of annulus 26). Upon immobilization of the portion of the circumference of annulus 26, a plurality of suture needles 14 positioned within support member 38a may be activated. Activation of suture needles 14 may extend the suture needles through the portion of the fibrous ring. Extending suture needles 14 through the portion of the fibrous ring may push portions of a suture 12a through the portion of the fibrous ring and into a plurality of needle openings positioned in support member 38b as depicted in FIG. 14. Suture needles 14 may then retract after extending through the portion of the fibrous ring. Retraction of suture needles 14 may be automated or manual. The portions of suture 12a extending through the portion of the fibrous ring may form suture loops as depicted in FIG. 13B. A second suture 12b may be threaded through the suture loops as depicted in FIG. 13C. Sutures 12a-b may be coupled to the trigones in FIG. 13D. Applying tension to second suture 12b after removal of retractor 32 may assist in reducing the size of mitral valve annulus 26 as depicted in FIG. 15.

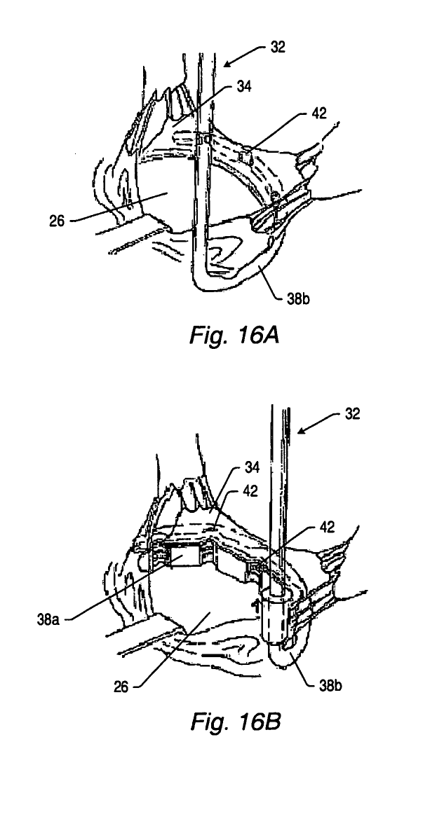

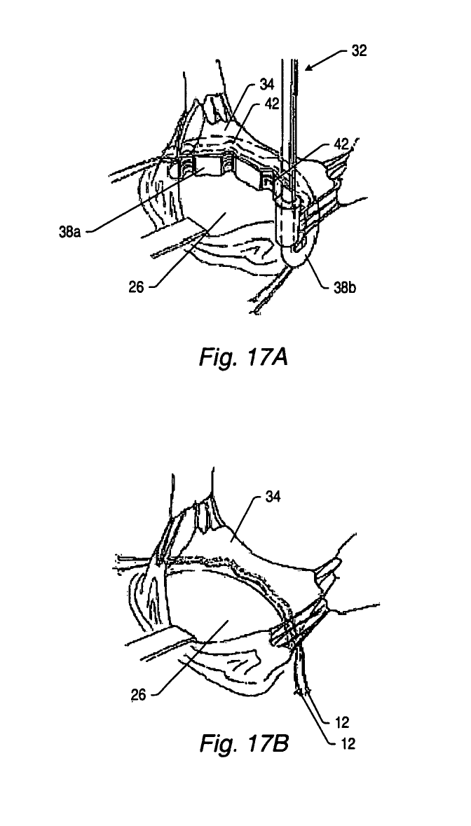

In certain embodiments, a method for reducing the size of mitral valve annulus 26 may combine the use of retractor 32 and a system of fasteners 36. FIGS. 16-17 depict an embodiment of a device combining the concept of retractor 32 and a system of fasteners 36. Retractor 32 may include two support members 38a-b as depicted in FIG. 16B. During a procedure the distal end of support members 38a-b may be positioned (e.g., rotated) into place adjacent posterior leaflet 34. Support member 38b may be positioned behind and/or below posterior leaflet 34 as depicted in FIG. 16A. Once support member 38b is positioned, support member 38a may be positioned substantially within annulus 26 and in front of posterior leaflet 34 as depicted in FIG. 16B. Moving support member 38a in such a manner may inhibit movement of portions of a circumference of annulus 26 (e.g., the fibrous ring of annulus 26). In some embodiments, support member 38a may be an elongated curved member coupled to support member 38b. Support member 38a may resemble something like a swingarm, as depicted in FIG. 16B, which is able to rotate around support member 38b. Support member 38a may be able to slide up and down along an axis of support member 38b. Upon immobilization of the portion of the circumference of annulus 26, one or more suture needles 14 coupled to sutures 12 may be passed through one or more conduits or channels (not shown). Suture needles 14 may pull sutures 12 through the conduits and consequently the fibrous ring of the portion of the circumference of annulus 26. Retractor 32 may then be removed leaving behind sutures 12 as depicted in FIG. 17B. Sutures 12 may be attached to trigones and/or tightened reducing the size of mitral valve annulus 26.

In some embodiments, a portion of support member 38b may include a plurality of depressions 42 as depicted in FIG. 17A. Support member 38a may include a plurality of depressions 42. Depressions 42 of support members 38a-b may be complementary to one another so that depressions 42 of support member 38a position portions of the fibrous ring of mitral valve annulus 26 into depressions 42 of support member 38b. Outer and/or inner surfaces of depressions 42 may include systems and/or artifacts (e.g., corrugation or teeth) to increase the coefficient of friction between surfaces of depressions 42 and tissue. Support member 38a may lock into place once positioned.

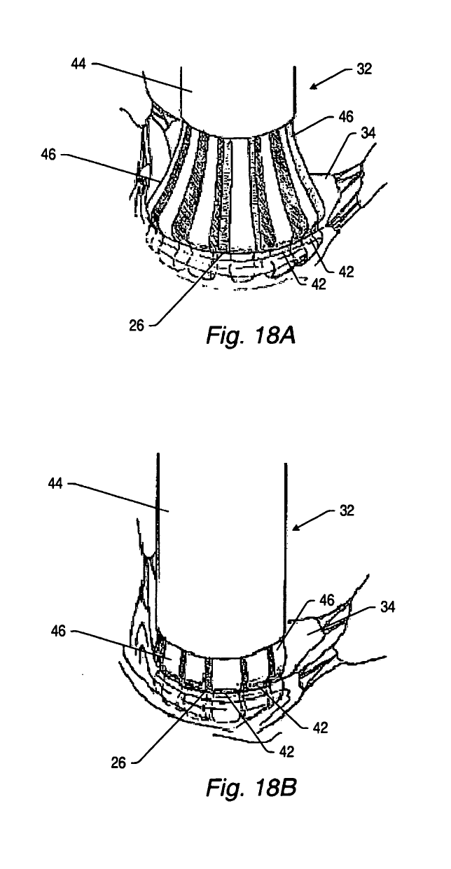

In certain embodiments, a method for reducing the size of mitral valve annulus 26 may include the use of retractor 32 as depicted in FIGS. 18A-B. Retractor 32 may include a conduit or elongated member 44. Retractor 32 may include a plurality of extendable members 46. Extendable members 46 may be formed from a substantially flexible material. The flexible material may include a “memory metal” capable of reassuming its original shape after distortion. Extendable members 46 may extend through conduit 44. In some embodiments, extendable members 46 may be coupled to a mechanism which extends through the length of conduit 44 allowing manipulation of extendable members 46 by a user. Conduit 44 may restrict a natural expansion of extendable members 46 while at least a majority of the extendable members reside in the conduit.

In some embodiments, extendable members 46 may be exposed by moving the extendable members relative to conduit 44 while positioning the distal end of the extendable members within mitral valve annulus 26. As extendable members 46 are exposed they will expand beyond the diameter of conduit 44. Expanding extendable members 46 may apply pressure to a portion of a circumference of mitral valve annulus 26, expanding the portion to a maximum diameter without damaging the portion.

In certain embodiments, the distal end of extendable members 46 may include depressions 42 in order to grip and/or position the fibrous ring of the portion more securely. Conduit 44 may be extended over a portion of extendable members 46 retracting the diameter of the expanded extendable members. The diameter of the fibrous ring of the portion gripped by the distal end of extendable members 46 may be reduced to a more desirable range, as depicted in FIG. 18B. Extendable members 46 may grip the fibrous ring of the portion using, for example, a vacuum and/or a mechanical feature. Mechanical features may include depressions 42 and/or coating and physical features (e.g., surface irregularities, teeth, or corrugation) that increase a coefficient of friction between the tissue and extendable members 46.

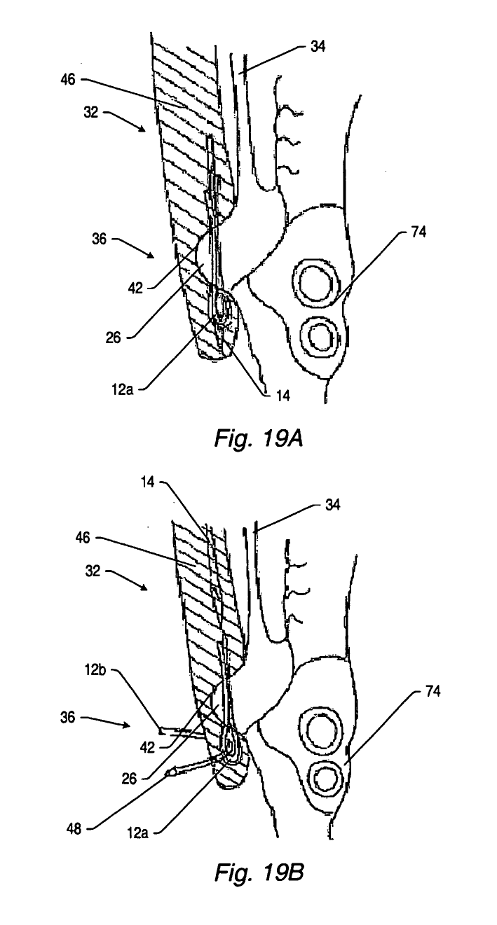

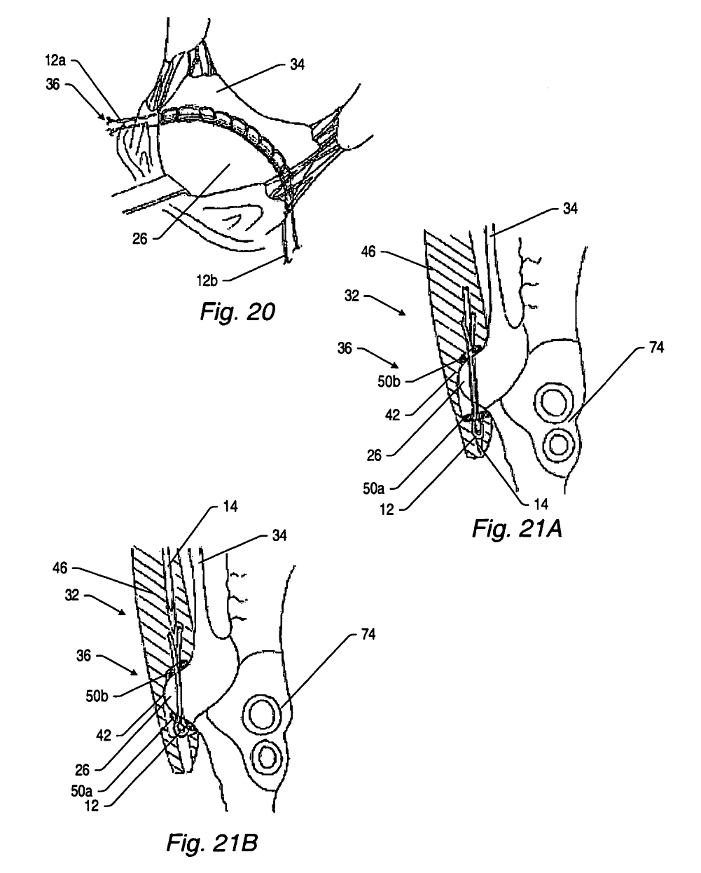

FIGS. 19A-B depict a cross sectional representation of an embodiment of a system of fasteners 36. In some embodiments system of fasteners 36 may be used in combination with retractor 32 (e.g., as depicted in FIGS. 18A-B). In certain embodiments, system of fasteners 36 may be used in combination with any other type of retractor 32 that falls within the scope of the spirit of the embodiments described. In some embodiments, suture needles 14 may pull sutures 12a through the fibrous ring forming portions of a circumference of mitral valve annulus 26. Suture needles 14 may pull sutures 12a through conduits formed in extendable members 46 and in and through the portions substantially positioned in depressions 42. In some embodiments, suture needles 14 may pull sutures 12a through openings between extendable members 46 and in and through the portions substantially positioned between depressions 42. Upon pulling sutures 12a through the portions, suture needles 14 may retract having formed loops of the sutures through the portions, as depicted in FIG. 19B. Upon formation of loops with sutures 12a radially oriented wire or suture needle 48 may guide or thread sutures 12b through at least a majority of the loops formed by sutures 12a. Removal of retractor 32 leaves sutures 12a and/or 12b behind available to couple to, for example, the trigones (as depicted in FIG. 20) to appropriately reduce the size of mitral valve annulus 26. It should be noted that a single suture tensioning technique is typically recommended but not exclusively useable.

In some embodiments, the retraction and extension of suture needles 14 and 48 may work in combination with the exposure and retraction of extendable members 46. For example suture needles 14 may extend and immediately retract as extendable members 46 reach a preset extension/exposure point Suture needle 48 may be activated as extendable members 46 reach a preset retraction point In certain embodiments, suture needles 14 and 48 may be activated independent of extendable members 46 by a user.

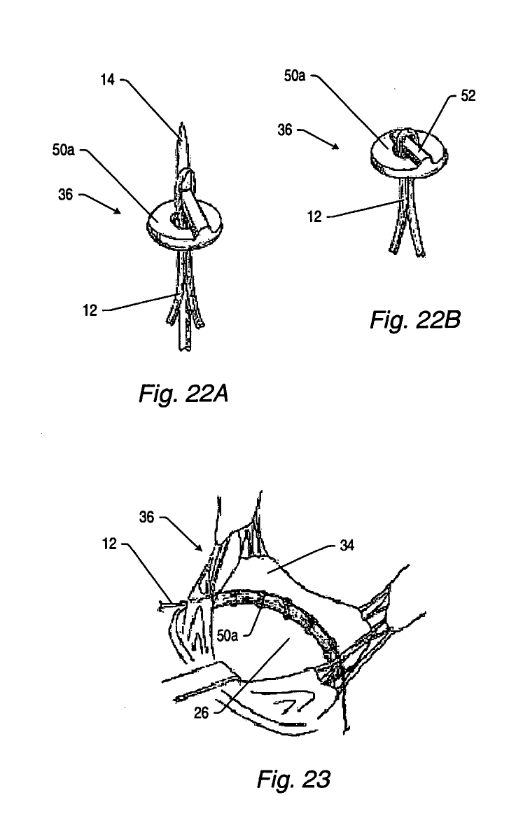

FIGS. 21-22 depict a representation of an embodiment of a system of fasteners 36. In some embodiments, suture needles 14 pull sutures 12 through the portion forming a loop which couples to coupler 50a after retraction of suture needles 14. In certain embodiments, there is no need for suture needle 48 or second suture 12b. FIGS. 22A-B depict a perspective view of an embodiment of coupler 50a Coupler 50a may be a substantially circular flat disk. Coupler 50a may be any other appropriate shape (e.g., square, rectangular, oval) and does not have to be particularly flat. Coupler 50a may include an opening extending through the coupler. Coupler 50a may include a coupling member 52. Coupling member 52 may be at one end of coupler 50a. Coupling member 52 may remain unattached on one end. During use, suture needle 14 pushes suture 12 through the opening in coupler 50a past the unattached end of coupling member 52. Upon retraction of suture needle 14, the loop formed by suture 12 encircles coupling member 52 as the coupling member returns to its original position. Coupling member 52 may inhibit extraction of suture 12 through the opening in coupler 50a. FIG. 23 depicts mitral valve annulus 26 after extraction of retractor 32 leaving sutures 12 behind available to couple to, for example, the trigones to appropriately reduce the size of mitral valve annulus 26.

In some embodiments, suture needle 14 may push suture 12 through the opening in a second coupler 50b before penetrating the fibrous ring forming mitral valve annulus 26, as shown in FIGS. 21A-B. Second coupler 50b may not include a coupling member 52. Second coupler 50b may assist in properly orienting portions of suture 12 forming loops through the fibrous ring. Second coupler 50b may inhibit portions of suture 12 from irritating and/or damaging tissue when the suture is tightened reducing the annulus of mitral valve annulus 26.

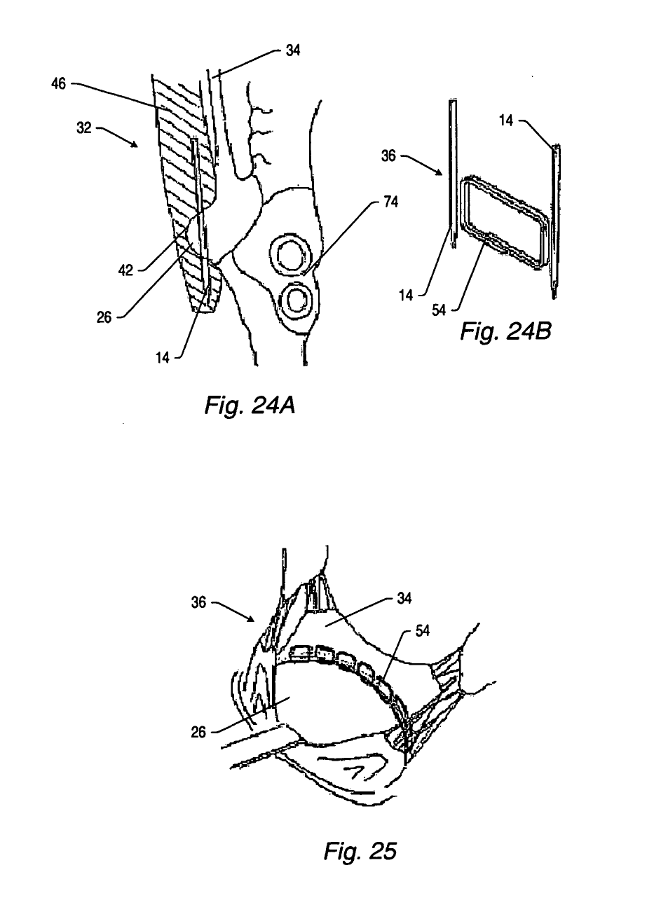

FIGS. 24-25 depict a representation of an embodiment of a system of fasteners 36 for reducing the size of a portion of a circumference of mitral valve annulus 26. In some embodiments, staples 54 may be used to assist in reducing mitral valve annulus 26. In some embodiments, retractor 32 may extend suture needles 14 from an original position between extendable members 46. Conduit 44 may be extended over a portion of extendable members 46 retracting the diameter of the expanded extendable members. The diameter of the fibrous ring of the portion gripped by the distal end of extendable members 46 may be reduced to a more desirable range.

During the reduction of a portion of a circumference of mitral valve annulus 26 suture needles 14 may remain positioned in the fibrous ring. Suture needles 14 may assist in gripping the fibrous ring as the diameter of the portion is reduced. Upon reduction of the diameter of the portion, extendable members 46 may be at least partially withdrawn while leaving suture needles 14 positioned in the fibrous ring. Advantages of withdrawing extendable members 46 while leaving suture needles 14 positioned may include allowing increased visibility and/or access to the portion of annulus 26 for a user. Upon withdrawal of extendable members 46, staples 54 may be positioned in the portion of annulus 26 between suture needles 14. Positioned staples 54 may be activated, wherein activation of the staples stabilizes the reduction of the portion. Activation of staples 54 may include distorting one or more parts of the staples, wherein the activated staples inhibit removal of the staples and/or enlargement of the now reduced portion. FIG. 24B depicts activated staples positioned between suture needles 14. Suture needles 14 may be withdrawn and retractor 32 removed by the user leaving the repaired annulus 26, as depicted in FIG. 25. In some embodiments, staples 54 may be positioned and activated by retractor 32 as a separate function of the retractor. In some embodiments, a different device dedicated to positioning and activating staples 54 may be used in combination with retractor 32.

In some embodiments, the function associated with suture needles 14 may be performed by staples 54. Inactivated staples 54 may be positioned in the fibrous ring forming portions of a circumference of mitral valve annulus 26. Retractor 32 may extend inactivated staples 54 from an original position between and/or at least partially in extendable members 46. Conduit 44 may be extended over a portion of extendable members 46 retracting the diameter of the expanded extendable members. The diameter of the fibrous ring of the portion gripped by the distal end of extendable members 46 may be reduced to a more desirable range. Inactivated staples 54 may assist in gripping the fibrous ring as the diameter of the portion is reduced. Upon reduction of the diameter of the portion, staples 54 may be activated as described inhibiting removal of the staples and/or enlargement of the now reduced portion.

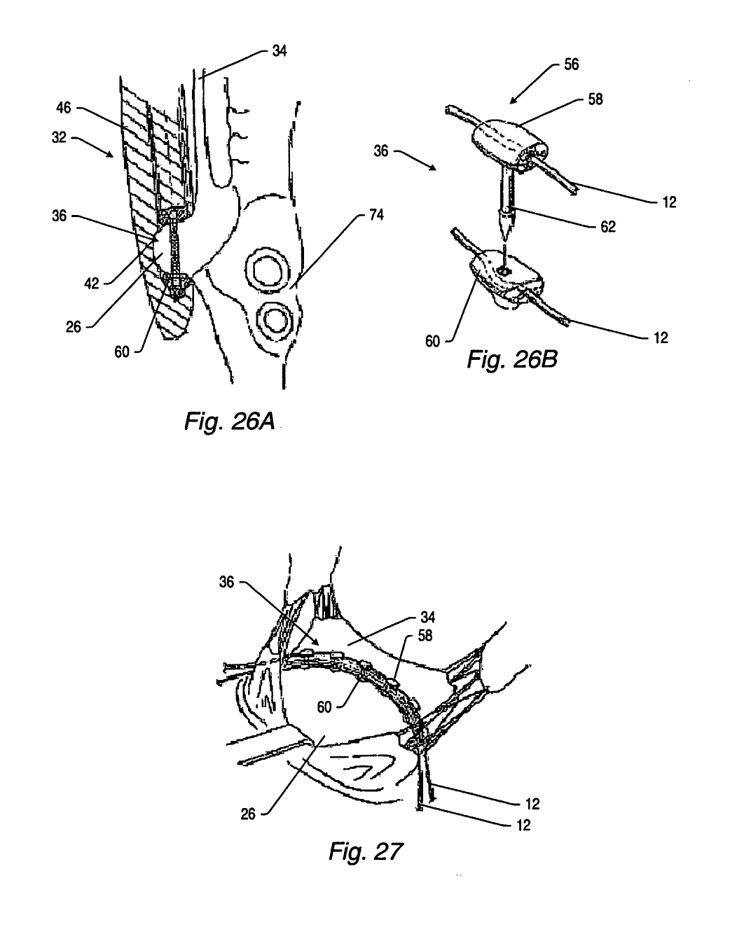

FIGS. 26-27 depict a representation of an embodiment of a system of fasteners 36 for reducing the size of a portion of a circumference of mitral valve annulus 26. In an embodiment, a system of fasteners 36 may include clips 56. In some embodiments, clips 56 may include two parts, referred to herein as fust clip 58 and second clip 60, as depicted in FIGS. 26A-B. With extendable members 46 extended and positioned so as to grip portions of the fibrous ring of the portion, clips 56 may be activated. Activating clips 56 may include positioning a part of first clip 58 through the fibrous ring and through a part of second clip 60. Activating clips 56 in such a way will couple first clip 58, second clip 60, and portions of the fibrous ring together. One or more sutures 12 may extend through clips 56 which may be attached to trigones to reduce the portion of the circumference of mitral valve annulus 26 upon removal of retractor 32, depicted in FIG. 27. Sutures 12 may be positioned through clips 56 before or after activation of the clips.

In certain embodiments, first clip 58 may include an opening extending through the body of the first clip. The opening extending through the body of first clip 58 may accommodate sutures 12. First clip 58 may include coupling member 62. Coupling member 62 may be positioned perpendicular relative to the opening extending through first clip 58. The distal end of coupling member 62 may include a point with greater diameter at the base relative to the point and remaining parts of the coupling member. Second clip 60 may include two openings extending through the body of the second clip. The first opening of second clip 60 may resemble the opening in first clip 58 accommodating sutures 12. The second opening of second clip 60 may include an initial (referring to the upper portion of the second clip) diameter smaller than the base of the point of coupling member 62, after which the diameter of the second opening may increase to a point substantially equal to or greater than the diameter of the base of the point. The depth of the second opening of second clip 60 may be at least as great as the point of coupling member 62. Upon activation of clips 56, the point of coupling member 62 passes through the second opening of second clip 60. Second clip 60 may be formed from semi-rigid material allowing the base of the point of coupling member 62 to pass through the smaller diameter initial opening of the second opening of the second clip. Upon the base of the point of coupling member 62 passing through the initial opening of the second opening of second clip 60, the base may inhibit coupling member 62 from exiting the second opening of second clip 60, effectively locking first clip 58 and the second clip together to the fibrous ring of the portion.

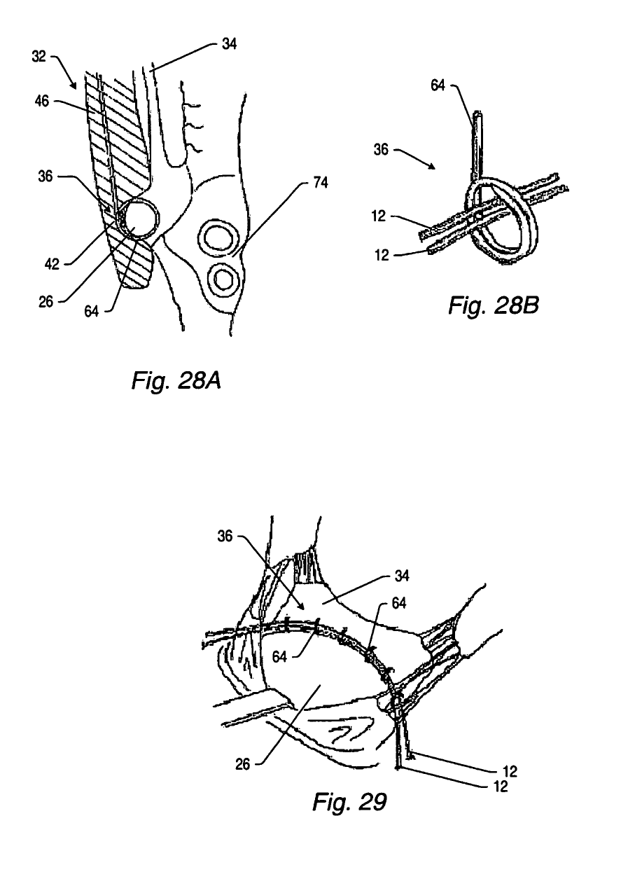

FIGS. 28-29 depict a representation of an embodiment of a system of fasteners 36 for reducing the size of a portion of a circumference of mitral valve annulus 26. In an embodiment, a system of fasteners 36 may include collars 64. In some embodiments, collars 64 may include a needle or pin formed of a semi-flexible material (e.g., a metal such as nitinol) as depicted in FIGS. 28A-B. In an inactivated, state collars 64 may be substantially straight or at least only slightly bent Inactivated collars 64 may initially be positioned in extendable members 46 before activation. With extendable members 46 extended and positioned so as to grip portions of the fibrous ring of the portion, collars 64 may be activated. Activating collars 64 may include using a mechanical force to push the collars out of an opening in extendable members 46. Collars 64 may be further forced into the fibrous ring of the portion. The end of the opening in collars 64 through which collars 64 exit upon activation may be slightly curved and complementary to an angle of curvature of depressions 42. Upon activating collars 64, an effect of the curvatures of the openings and depressions 42 may result in the collars forming at least one complete loop penetrating the fibrous ring of the portion, as depicted in FIG. 28A. Activated collars 64 may encircle one or more sutures 12 coupling the sutures to the fibrous ring of the portion. One or more sutures 12 extending through collars 64 may be attached to trigones to reduce the portion of the circumference of mitral valve annulus 26 upon removal of retractor 32, depicted in FIG. 29. Sutures 12 may be positioned through collars 64 before and/or after activation of the collars.

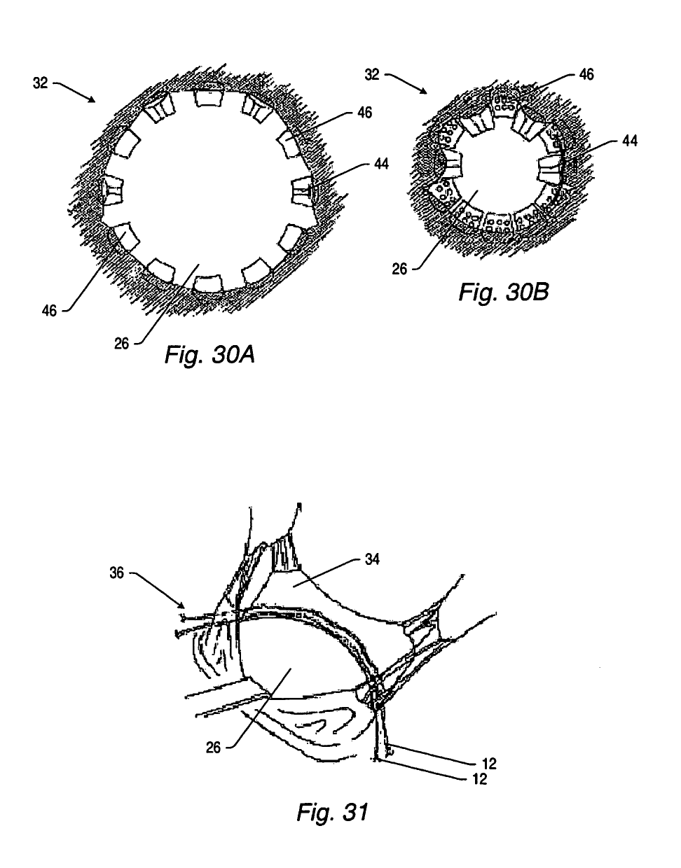

FIGS. 30-31 depict a representation of an embodiment of a system for reducing the size of a portion of a circumference of mitral valve annulus 26. In some embodiments, system of fasteners 36 may include suture needles 14 and sutures 12. In certain embodiments, suture needle 14 may include a curved suture needle. In an embodiment, extendable members 46 may not include depressions 42. At least some of extendable members 46 may include suction ports 44. Suction ports 44 may extend through extendable members 46. Suction ports 44 may be coupled to a vacuum producing mechanism capable of drawing a controlled vacuum through the suction ports. Drawing a vacuum through suction ports 44 may further delineate portions of the fibrous ring forming mitral valve annulus 26. Drawing a vacuum through suction ports 44 may facilitate the use of extendable members 46 as suture guides. In certain embodiments, only some of extendable members 46 may include suction ports 44, thus forming a pattern of alternating extendable members 46 which include suction ports 44, as depicted in FIGS. 30A-B. With extendable members 46 extended and positioned adjacent the fibrous ring of the portion, a vacuum may be applied through suction ports 44, to couple extendable members 46, including the suction ports, to the fibrous ring. Conduit 44 may be extended over a portion of extendable members 46 retracting the diameter of the expanded extendable members. The diameter of the fibrous ring of the portion gripped by the distal end of extendable members 46 may be reduced to a more desirable range, as depicted in FIG. 30B. Extendable members 46 which include suction ports 44 may be further retracted relative to the extendable members without suction ports. Further retracting extendable members 46 which include suction ports 44 may deform the fibrous ring of the portion forming depressions 42 as depicted in FIG. 30B.

In certain embodiments, a curved suture needle 14 and sutures 12 may be passed through the fibrous ring of the deformed portion along extendable members 46. In some embodiments, suture needle 14 and sutures 12 may not pass through the fibrous ring forming depressions 42. In some embodiments, one or more sutures 12 extending through the fibrous ring of the portion may be attached to trigones to reduce the portion of the circumference of mitral valve annulus 26 upon removal of retractor 32, depicted in FIG. 31.

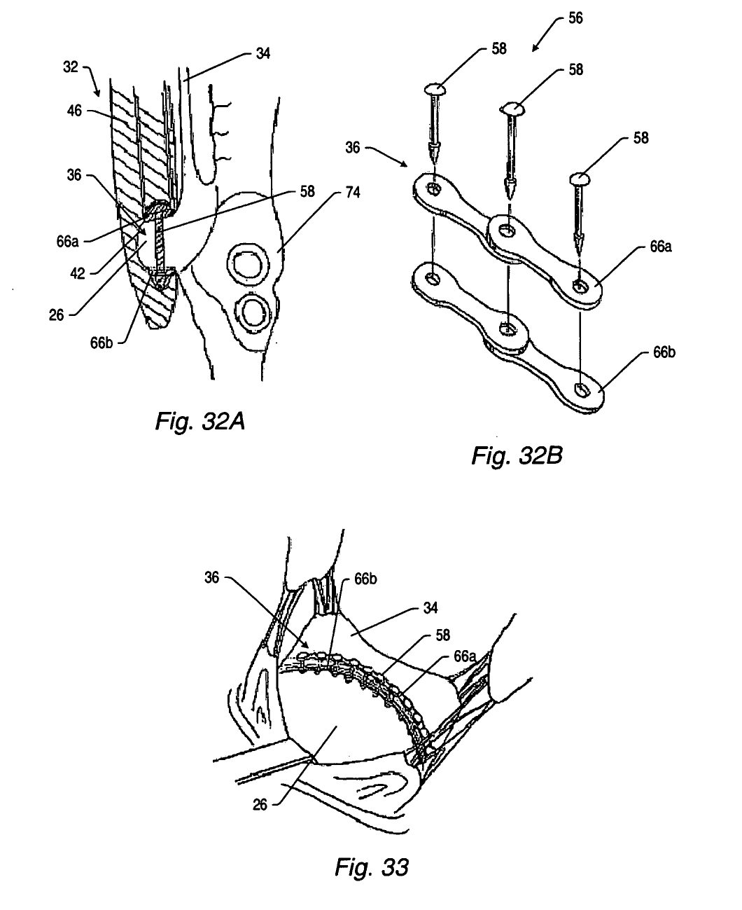

FIGS. 32-33 depict a representation of an embodiment of a system of fasteners 36 for reducing the size of a portion of a circumference of mitral valve annulus 26. In an embodiment, system of fasteners 36 may include clips 56. In some embodiments, clips 56 may include three parts, referred to herein as first clip 58, first plate 66a, and second plate 66b, as depicted in FIGS. 32A-B. With extendable members 46 extended and positioned so as to grip portions of the fibrous ring of the portion, clips 56 may be activated. Activating clips 56 may include positioning a part of first clip 58 through a first opening of first plate 66a, the fibrous ring, and through first opening of second plate 66b. Activating clips 56 in such a way will couple first clip 58, first plate 66a, second plate 66b, and portions of the fibrous ring together. Conduit 44 may be extended over a portion of extendable members 46 to retract the diameter of the expanded extendable members. The diameter of the fibrous ring of the portion gripped by the distal end of extendable members 46 may be reduced to a more desirable range. The fibrous ring of the portion may be reduced to a point wherein a second set of openings of first plate 66a and second plate 66b are substantially in alignment. Upon substantial alignment of the second set of openings of first plate 66a and second plate 66b, a second set of first clips 58b may be positioned through the second opening of first plate 66a, the fibrous ring, and through the second opening of second plate 66b. Positioning the second set of first clips 58b through the second opening of first plate 66a, the fibrous ring, and through the second opening of second plate 66b will couple first plate 66a, the fibrous ring, and second plate 66b together, as well as coupling first plates 66a to one another and coupling second plates 66b to one another. Coupling first plates 66a to one another, as well as coupling second plates 66b to one another may inhibit expansion of the now reduced diameter of the fibrous ring of the portion upon removal of retractor 32, as depicted in FIG. 33.

In certain embodiments, first clip 58 may include a coupling member 62. The distal end of coupling member 62 may include a point with a greater diameter at the base relative to the point and remaining parts of the coupling member. First plate 66a and second plate 66b may include two openings extending through the body of the plates. The openings of first plate 66a and second plate 66b may include an initial (referring to the upper surface of the plates) diameter smaller than the base of the point of coupling member 62, after which the diameter of the openings may increase to a point substantially equal to or greater than the diameter of the base of the point The depth of the openings of second plate 66b may be at least as great as the point of coupling member 62. Upon activation of clips 56, the point of coupling member 62 passes through the openings of first plate 66a and second plate 66b. First plate 66a and second plate 66b may be formed from semi-rigid material allowing the base of the point of coupling member 62 to pass through the smaller diameter initial opening of the openings of the plates. Upon the base of the point of coupling member 62 passing through the initial opening of the openings of first plate 66a and second plate 66b, the base may inhibit coupling member 62 from exiting the openings of the plates effectively coupling first clip 58 and the plates together to the fibrous ring of the portion.

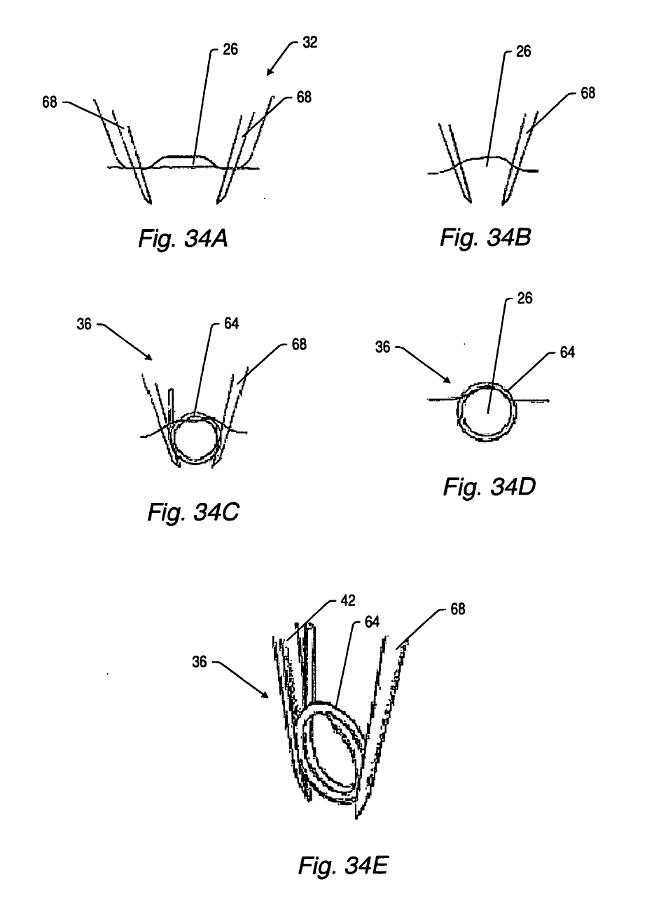

FIGS. 34A-E depict a representation of an embodiment of a system of fasteners 36 for reducing the size of a portion of a circumference of mitral valve annulus 26. In an embodiment, system of fasteners 36 may include collars 64. In some embodiments, retractor 32 may include a pair of needles or pincers 68, as depicted in FIG. 34E. In some embodiments, collars 64 may include a needle or pin formed of a semi-flexible material (e.g., a metal such as nitinol) as depicted in FIGS. 34C-E. In an inactivated state, collars 64 may be substantially straight or at least only slightly bent.

The distal end of pincers 68 may be inserted in the tissue and/or fibrous ring of the portion of the circumference of mitral valve annulus 26, as depicted in FIG. 34A. The distal end of pincers 68 may be moved towards one another. Moving the distal end of pincers 68 towards one another will pinch or gather any intervening tissue together and reduce the size of the portion of the circumference as depicted in FIG. 34B. The distal end of collars 64 may be inserted in to the gathered tissue and the collars activated. Activating collars 64 may include using a mechanical force to push the collars out of depression 42 in the distal end of pincers 68. Collars 64 may be further forced into the fibrous ring of the portion. The end of depression 42 in pincers 68 through which collars 64 exit upon activation may be slightly curved and complementary to the depression on the face of the second pincer. Upon activating collars 64, an effect of the curvatures of depressions 42 will result in the collars forming at least one complete loop penetrating the fibrous ring of the portion as depicted in FIG. 34C. Activated collars 64 may encircle the gathered tissue of the fibrous ring of the portion. Encircling the gathered tissue of the fibrous ring of the portion with collars 64 may inhibit the reduced portion of the circumference from expanding to its original diameter upon removal of pincers 68. Pincers 68 may be retracted from the fibrous ring leaving activated collars 64 in position as depicted in FIG. 34D.

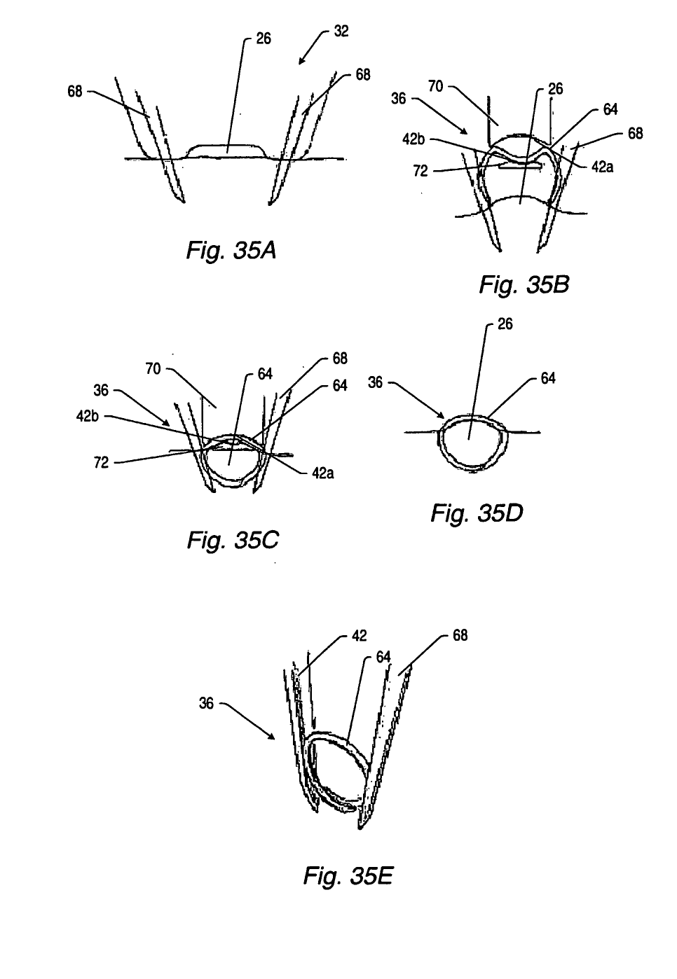

FIGS. 35A-E depict a representation of an embodiment of a system of fasteners 36 for reducing the size of a portion of a circumference of mitral valve annulus 26. In an embodiment, system of fasteners 36 may include collars 64. In some embodiments, retractor 32 may include a pair of needles or pincers 68 as depicted in FIG. 35E. In some embodiments, collars 64 may include a double ended needle formed of a semi-flexible material (e.g., a metal such as nitinol). In an inactivated state, collars 64 may be substantially shaped like a distorted “M”. An upper inward curvature of collars 64 will allow an area contained substantially within the collars to remain unchanged when the collars are activated. As the area within collars 64 is reduced by forcing the ends of the collars together, the decrease in area is substantially offset by the reduction and/or elimination of the upper inward curvature of the collars. An advantage of a design of this type may include that activation of collars 64 may not further decrease an already reduced size of the portion of the circumference.

The distal end of pincers 68 may be inserted in the tissue and/or fibrous ring of the portion of the circumference of mitral valve annulus 26 as depicted in FIG. 35A. The distal end of pincers 68 may be moved towards one another. Moving the distal end of pincers 68 towards one another will pinch or gather any intervening tissue together and reduce the size of the portion of the circumference as depicted in FIG. 35B. The distal end of collars 64 may be inserted in to the gathered tissue and the collars activated. Activating collars 64 may include using a mechanical force to push the collars out of depression 42 in the distal end of pincers 68. Collars 64 may be further forced into the fibrous ring of the portion. In certain embodiments, a driver 70 may be used to assist a user to insert collar 64 in the fibrous ring of the portion. In some embodiments, the end of depression 42 in pincers 68 through which collars 64 exit upon activation may be slightly curved and complementary to the depression on the face of the second pincer. Upon activating collars 64, an effect of the curvatures of depressions 42 will result in the collars forming at least one complete loop penetrating the fibrous ring of the portion, as depicted in FIG. 35C. Activated collars 64 may encircle the gathered tissue of the fibrous ring of the portion. Encircling the gathered tissue of the fibrous ring of the portion with collars 64 may inhibit the reduced portion of the circumference from expanding to its original diameter upon removal of pincers 68. Pincers 68 may be retracted from the fibrous ring and leave activated collars 64 in position, as depicted in FIG. 35D.

In some embodiments, a driver 70 may be used to assist a user to insert collar 64 in the fibrous ring of the portion. The distal end of driver 70 may include depression 42a Depression 42a may be complementary to an upper curvature of activated collar 64. Driver 70 may include base 72. Base 72 may include depression 42b. Depression 42b may be complementary to an upper curvature of inactivated collar 64. Depression 42b may function as a seat for the upper curvature of inactivated collar 64. During use the upper curvature of inactivated collar 64 may be positioned in the area between depression 42a and depression 42b of base 72. A user may employ driver 70 to apply a force on the upper curvature of inactivated collar 64 by bringing depression 42a and depression 42b of base 72 towards one another. Applying a force on the upper curvature of inactivated collar 64 may invert depression 42b of base 72 as the distal ends of collar 64 move together to form activated collar 64.

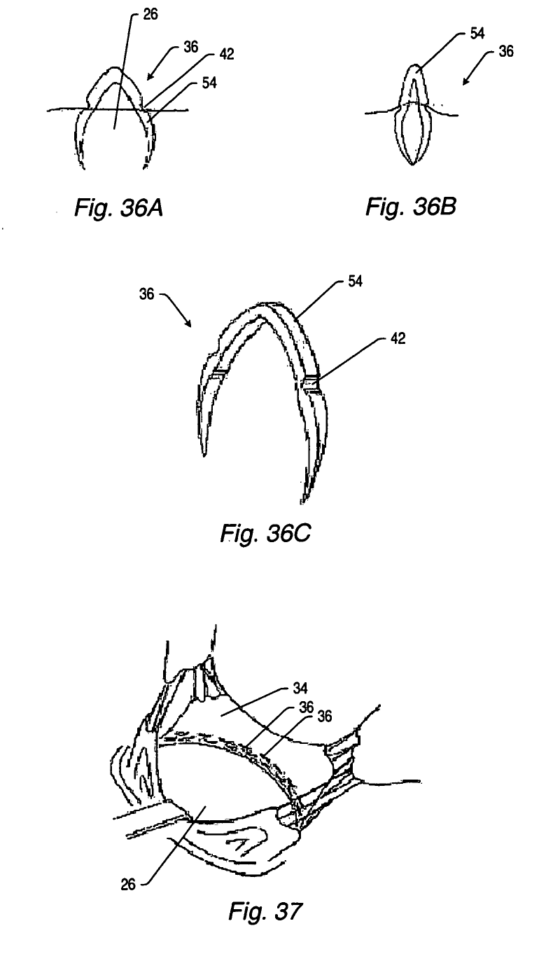

FIGS. 36A-C depict a representation of an embodiment of a system of fasteners 36 for reducing the size of a portion of a circumference of mitral valve annulus 26. In an embodiment, system of fasteners 36 may include staples 54. In some embodiments, retractor 32 may include a pair of pincers. In some embodiments, staples 54 may include a double ended needle formed of a semi-flexible material (e.g., a metal such as nitinol) In an inactivated state, staples 54 may be substantially shaped like an inverted “V”. The distal ends of staples 54 may come to a point facilitating insertion in the portion of the circumference of mitral valve annulus 26. The distal end of inactivated staples 54 may be inserted into tissue up to just below depressions 42. The distal end of pincers 68 may grasp a part of inactivated staples 54. Depressions 42 may function as a point at which the distal end of pincers 68 more effectively grasp staples 54. Upon grasping inactivated staples 54 with pincers 68, the pincers may be used to activate the staples. Pincers 68 may activate inactivated staples 54 by applying pressure to either side of the staples. Activating staples 54 may gather or compress the fibrous ring of the portion located within the area between the distal ends of the staples. Compressing the fibrous ring of the portion will reduce the size of the portion of the circumference of mitral valve annulus 26.

FIG. 37 depicts a perspective view of a mitral valve annulus after positioning a system of annulus reducing fasteners such as those depicted in FIGS. 34-36. In some embodiments, fasteners 36 may be place in two or more rows. The fasteners in the rows may be staggered. In certain embodiments, fasteners 36 may be positioned behind posterior leaflet 34.

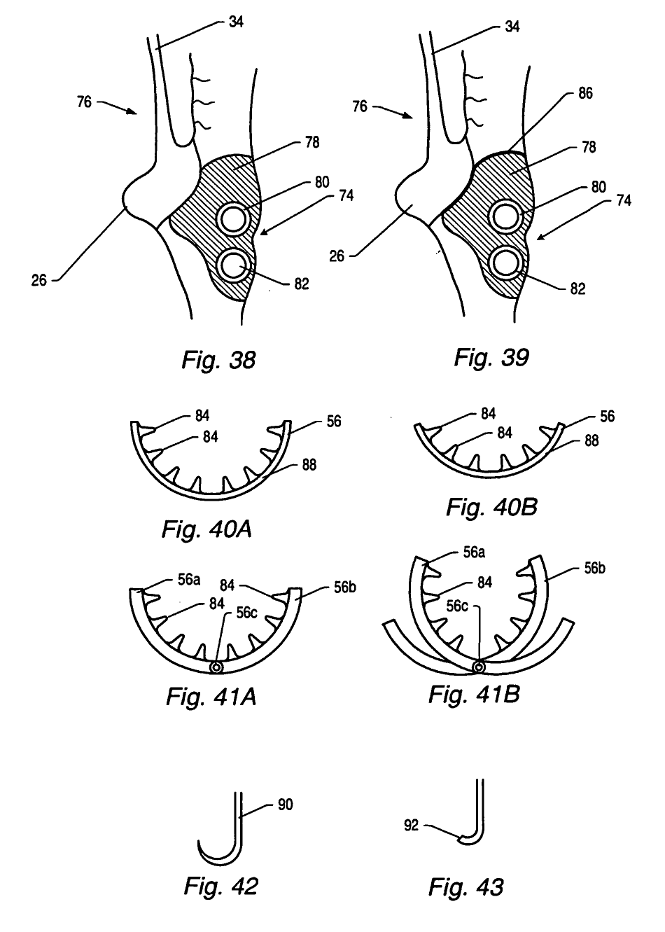

In some embodiments, the ability of a user to quickly and accurately repair a dilation of a mitral valve annulus 26 may be addressed. A coronary sinus region 74 proximate to mitral valve annulus 26 (depicted in FIG. 38) may be accessed. Coronary sinus 74 predominantly drains left ventricle 76 and receives approximately 85% of the coronary venous blood. Coronary sinus 74 lies within the posterior atrioventricular (AV) groove and empties into the right atrium. In some embodiments, a method may include making incision 86 of fatty tissue 78 of the AV groove along the posterior annulus of mitral valve 26, as depicted in FIG. 39. Incision 86 may either be made above great cardiac vein 80 or below circumflex artery 82.

Great cardiac vein 80 runs parallel to the circumflex branch in the left AV groove and continues as the coronary sinus 74. Great cardiac vein 80 is a large vein with two branches, the left coronary vein and the right coronary vein. Vein 80 commences at the tip of the heart and ascends along the heart to the base of the ventricles. Great cardiac vein 80 then curves left to the back portion of the heart and opens into the left coronary sinus, which is about 1 inch in length and terminates in the right atrium near the inferior vena cava. Care should be taken when placing a device in the AV groove of the heart as there is a possibility of impairing or occluding the circumflex artery which is proximate to the AV groove.

In certain embodiments, a device, (such as a shaped clip 56 as depicted in FIGS. 40A, 40B, 41A, and 41B) may be placed through an incision and along a portion of great cardiac vein 80 or within the great cardiac vein. Shaped clip 56 may be adjustable. Shaped clip 56 may be deformed in order to reduce the size of mitral valve annulus 26.

In some embodiments, a method for reducing a size of mitral valve annulus 26 may include incising a portion of a fatty tissue 78 in an AV groove proximate to mitral valve annulus 26. A device may be positioned through the incision and proximate to a portion of a great cardiac vein 80. The device may include a plurality of attachment members 84. The method may include coupling attachment members 84 to a portion of mitral valve annulus 26. The method may include deforming the device. In some embodiments, mitral valve annulus 26 and a surrounding area may be observed under echocardiography. If negative results are indicated from the echocardiography (such as mitral regurgitation), the device may be further deformed. Deforming the device may reduce the size of mitral valve annulus 26. The device may be continually adjusted until such time as no more negative results are indicated. In some embodiments, a method for reducing a size of mitral valve annulus 26 may include positioning at least one object along a portion of the annulus, and causing the at least one object to reduce the portion of the annulus.

In some embodiments, a method for reducing a size of mitral valve annulus 26 may include reducing a size of an annulus by a device. The user may wish to place the device directly into great cardiac vein 80. There are certain advantages to this type of procedure which may include ease of placement of the device as well as mitigating the risk of occluding or constricting any arteries. Accordingly, a method for reducing a size of mitral valve annulus 26 may include incising a portion of great cardiac vein 80. The method may include positioning a device through the incision. The method may include deforming the device. Deforming the device may reduce the size of the annulus.

In some embodiments, a device, may include shaped clip 56. Shaped clip 56 may be adapted to reduce a size of a mitral valve annulus 26. Shaped clip 56 may include an approximately semicircular adjustable body 88, and one or more attachment members 84 coupled to one side of the body (see FIG. 40A). Shaped clip 56 may be adapted to be deformed (see FIG. 40B). Deformation of shaped clip 56 may cause an engagement of annulus 26 by attachment members 84 reducing the size of the annulus.

In some embodiments, clips or devices may be used to reduce the size of mitral valve annulus 26. Clip 56 is depicted in an open position in FIG. 41A and an adjusted position in FIG. 41B. Clip 56 may include first portion 56a, second portion 56b, and third portion 56c. First portion 56a and second portion 56b may be slidably attached to third portion 56c. Third portion 56c may be adapted to secure first portion 56a and second portion 56b. First portion 56a and/or second portion 56b may include one or more attachment members 84. Attachment members 84 may be positioned on similar sides of first portion 56a and second portion 56b. The radius (R1) of clip 56 in the open position is greater than the radius (R2) of the clip in the adjusted position. Clip 56 may be formed of non-rigid material, so the clip may flex along with annulus 26.

In some embodiments, during a procedure to reduce a size of mitral valve annulus 26, some of the portions of the heart described above (such as the AV groove) may be difficult to visually observe. As such, specially shaped scalpel 90, as depicted in FIG. 42, may be used to blindly incise into the AV groove fat pad. In some embodiments, scalpel 90 comprises a base, a curved portion extending from the base, and a blade extending from the curved portion, wherein the blade is adapted to blindly incise the AV groove. The blade may be a V-shaped blade and may include various materials, other shapes, lengths, and/or thicknesses.

In some embodiments, to assist the user in making such an incision, a reflective device or surface, such as mirror 92 as depicted in FIG. 43, may be used. In some embodiments, mirror 92 includes a base, and a mirror extending from the base. The mirror may be adapted to provide a view of the incision area of the AV groove. Such a device may be hand held, fixed in a certain position, or moveably controlled.

In summary, surgical methods and devices to repair dilation of the mitral valve annulus are disclosed. These methods and devices produce improved results in patients, and are much less expensive and easier to use than prior art methods and devices.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method for reducing a size of a mitral valve annulus, comprising:
positioning at least a portion of a retractor substantially adjacent a portion of a circumference of the annulus;
coupling one or more of a plurality of extendable members of the retractor to the portion of the circumference;
reducing the diameter of an opening in the distal end of the retractor, wherein the opening is at least partially defined by the portion of the retractor coupled to the portion of the circumference, and wherein reducing the diameter of the opening in the distal end of the retractor reduces the diameter; and
positioning at least a portion of one or more fasteners in the portion of the circumference to inhibit deformation of the reduced diameter of the portion of the circumference of the mitral valve annulus.

2. (canceled)

3. The method claim 1, wherein the retractor comprises:
a conduit, wherein the plurality of extendable members are at least partially positionable in the conduit, wherein the plurality of extendable members at least partially defines the opening in the distal end of the retractor, wherein extending the distal ends of the plurality of extendable members out of the conduit increases the corresponding diameter of the predetermined shape formed by the plurality of extendable members, and wherein retracting the distal ends of the plurality of extendable members out of the conduit decreases the corresponding diameter of the predetermined shape formed by the plurality of extendable members; and
a depression positioned towards the distal end of the outer surface of at least some of the plurality of extendable members;
wherein positioning the portion of the retractor adjacent the annulus comprises positioning the distal end of the retractor with retracted extendable members substantially in the annulus and extending the extendable members until the portion of the circumference of the annulus is positioned at least substantially in the depressions.

4. The method of claim 1, further comprising providing a vacuum through one or more openings positioned in one or more of the plurality of extendable members.

5. (canceled)

6. (canceled)

7. The method of claim 4, further comprising positioning a suture through the portions of the circumference coupled to the vacuum openings of the extendable members.

8. (canceled)

9. (canceled)

10. The method of claim 1, further comprising adhering the portion of the circumference to a depression positioned towards the distal end of the outer surface of at least some of the plurality of extendable members, wherein the depression comprises one or more surface irregularities to increase the coefficient of friction between the surface of the depression and the portion of the circumference.

11. (canceled)

12. The method of claim 1, further comprising extending one or more suture needles positioned in a conduit in one or more of the extendable members through a depression positioned towards the distal end of the outer surface of the extendable member and the portion of the circumference of the annulus positioned in the depression, wherein extending the suture needles through the depression and the portion of the circumference comprises forming a suture loop extending through the portion of the circumference.

13. The method of claim 12, further comprising positioning a second suture through the plurality of suture loops.

14. The method of claim 1, further comprising extending one or more suture needles positioned in a conduit in one or more of the extendable members through a depression positioned towards the distal end of the outer surface of the extendable member, the portion of the circumference of the annulus positioned in the depression, and an opening in a coupler positioned toward the distal end of the depression, wherein extending the suture needles through the depression, the portion of the circumference, and the opening in the coupler comprises forming a suture loop extending through the portion of the circumference, the opening in the coupler and around a coupling member, and wherein the coupling member inhibits the suture loop from retracting through the opening in the coupler.

15. The method of claim 14, wherein the formed suture loop extends through a second opening in a second coupler positioned toward the proximal end of the depression.

16. The method of claim 15, further comprising inhibiting movement of the proximal end of the suture loop with the second coupler.

17. The method of claim 1, further comprising extending one or more coupling members of a first clip positioned in a conduit in one or more of the extendable members through a depression positioned towards the distal end of the outer surface of the extendable member, the portion of the circumference of the annulus positioned in the depression, and an opening in a second clip positioned toward the distal end of the depression.

18. The method of claim 17, further comprising inhibiting retraction of the distal end of the coupling member with the opening in the second clip.

19. (canceled)

20. (canceled)

21. The method of claim 1, further comprising activating one or more collars comprising extending one or more inactivated collars positioned in a conduit in one or more of the extendable members through a depression positioned towards the distal end of the outer surface of the extendable member and the portion of the circumference of the annulus positioned in the depression, wherein the depression facilitates activation of the extending inactivated collars, wherein the activated collars comprise a loop extending through the circumference of the annulus.

22. The method of claim 21, wherein the loop of the activated collars extends around one or more sutures.

23. The method of claim 1, further comprising extending one or more first clips positioned in a conduit in one or more of the extendable members through a first opening in a first plate positioned toward the proximal end of a depression, the depression positioned towards the distal end of the outer surface of the extendable member, the portion of the circumference of the annulus positioned in the depression, and a first opening in a second plate positioned toward the distal end of the depression, wherein extending the first clip couples the first plate, the portion of the circumference of the annulus, and the second plate to each other.

24. The method of claim 23, further comprising positioning one or more first clips through a second opening in the first plate, the portion of the circumference of the annulus, and a second opening in the second plate, wherein extending the first clip couples the first plate, the portion of the circumference of the annulus, and the second plate to each other.

25. (canceled)

26. A method for reducing a size of a mitral valve annulus, comprising:
positioning at least a portion of two or more support members of a retractor substantially adjacent a posterior leaflet;
using the support members to position the posterior leaflet such that a portion of the mitral valve annulus is substantially exposed;
positioning at least a portion of one or more fasteners in the portion of the circumference; and
activating the fasteners to reduce the diameter of the portion of the circumference when activated.

27. The method of claim 26, further comprising:
positioning a first support member and a second support member substantially on a first side of the posterior leaflet, and wherein the second support member is adjacent the posterior leaflet;
positioning a third support member on a second side of the posterior leaflet, wherein the second side is opposite the first side, and wherein the second side is adjacent the mitral valve annulus; and
moving the second support member toward the third support member such that the posterior leaflet is inhibited from moving, and moving the second and third support members away from the first support member such that at least a portion of the posterior leaflet is pulled away from the mitral valve annulus to use the support members to position the posterior leaflet.

28-36. (canceled)

37. The method of claim 26, wherein the fasteners comprises one or more sutures.

38-42. (canceled)

43. A method for reducing a size of a mitral valve annulus, comprising:
positioning at least a portion of an inactivated fastener substantially in a portion of a circumference of the annulus;
activating the inactivated fastener reducing the diameter of the portion of the circumference, wherein the activated fastener inhibits deformation of the reduced diameter of the portion of the circumference of the mitral valve annulus.

44-70. (canceled)
